SEQUENCE LISTING (2 sequences)

SEQ ID NO: 1 - protein, 107 aa, Artificial
  hBU12 light chain variable region
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKR

SEQ ID NO: 2 - protein, 120 aa, Artificial
  hBU12 heavy chain variable region
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSS

(19) United States
(12) Patent Application Publication
(10) Pub. No.: US 2018/0028681 A1
(43) Pub. Date: Feb. 1, 2018

(54) COMBINATION THERAPY USING A CD19-ADC AND VINCRISTINE

(71) Applicant: SEATTLE GENETICS, INC., Bothell, WA (US)

(72) Inventors: Che-Leung Law, Shoreline, WA (US); Ivan Stone, Everett, WA (US)

(73) Assignee: SEATTLE GENETICS, INC., Bothell, WA (US)

(21) Appl. No.: 15/550,126

(22) PCT Filed: Feb. 12, 2016
(86) PCT No.: PCT/US16/17721

§ 371 Date: Aug. 10, 2017

Related U.S. Application Data
(60) Provisional application No. 62/115,381, filed on Feb. 12, 2015.

(51) Int. Cl. A61K 47/68; A61K 31/475; A61K 31/573; A61K 31/675; A61K 31/704; C07K 16/28; A61K 38/06

(52) U.S. Cl. CPC A61K 47/6867; C07K 16/2803; A61K 31/475; A61K 38/06; A61K 31/675; A61K 31/704; A61K 31/573; C07K 2317/24

(57) ABSTRACT

This invention relates to treatment of acute lymphoblastic leukemia.

### CROSS-REFERENCES TO RELATED APPLICATIONS

NOT APPLICABLE

### FIELD OF THE INVENTION

This invention relates to treatment of acute lymphoblastic leukemia.

### BACKGROUND OF THE INVENTION

CD19 is a member of the immunoglobulin superfamily. See, e.g., Tedder & Isaacs, J Immunol, 143:712-717 (1989) and Del Nagro et al., Immunol Res, 31:119-131 (2005). It is a B cell-specific marker not known to be expressed by any cell outside of the B lineage. CD19 expression is maintained upon malignant transformation, thus, CD19 is found on malignant cells in the majority of patients with B-cell leukemia or non-Hodgkin lymphoma. See, e.g., Nadler et al., J Immunol, 131:244-250 (1983); Anderson et al., Blood, 63:1424-1433 (1984); and Scheuermann & Racila, Leuk Lymphoma, 18:385-397 (1995).

SGN-CD19A is a CD19-directed antibody-drug conjugate (ADC) consisting of three components: 1) the humanized antibody hBU12, specific for human CD19, 2) the microtubule disrupting agent, monomethyl auristatin F (MMAF), and 3) a stable linker, maleimidocaproyl, that covalently attaches MMAF to hBU12. The proposed mechanism of action (MOA) is initiated by SGN-CD19A binding to CD19 on the cell surface followed by internalization of the ADC. Upon trafficking to lysosomes, the delivered drug (cysmcMMAF) is released through proteolytic degradation of the antibody carrier. Binding of the released drug to tubulin disrupts the microtubule network, leading to cell cycle arrest and apoptosis.

SGN-CD19A activity has recently been assessed in a phase 1 clinical trial for treatment of patients with B-linage acute lymphoblastic leukemia (B-ALL or ALL). Howver, improvements are needed in cancer therapy. The present invention solves this and other problems.

### BRIEF SUMMARY OF THE INVENTION

In one aspect, the present disclosure provides a method of treating a subject with acute lymphoblastic leukemia (ALL), by administering a drug combination consisting essentially of a CD19 antibody drug conjugate (CD19-ADC) and vincristine. The CD19-ADC is preferably SGN-CD19A, i.e., a humanized hBU12 antibody conjugated to a maleimidocaproyl monomethyl auristatin F (mcMMAF) molecule. In one embodiment, the subject has relapsed or refractory ALL. In another embodiment, one of the following drugs is also administered to the subject: cyclophosphamide, doxorubicin, or dexamethasone. In another embodiment, the following three drugs are also administered to the subject: cyclophosphamide, doxorubicin, and dexamethasone. In a further embodiment, the CD19-ADC is administered at a dosage between 0.5 and 6.0 mg/kg.

In one aspect, the present disclosure provides a method of treating a subject with acute lymphoblastic leukemia (ALL), by administering a drug combination consisting essentially of a CD19 antibody drug conjugate (CD19-ADC) and doxorubicin. The CD19-ADC is preferably SGN-CD19A, i.e., a humanized hBU12 antibody conjugated to a maleimidocaproyl monomethyl auristatin F (mcMMAF) molecule. In one embodiment, the subject has relapsed or refractory ALL. In another embodiment, one of the following drugs is also administered to the subject: cyclophosphamide, vincristine, or dexamethasone. In another embodiment, the following three drugs are also administered to the subject: cyclophosphamide, vincristine, and dexamethasone. In a further embodiment, the CD19-ADC is administered at a dosage between 0.5 and 6.0 mg/kg.

In one aspect, the present disclosure provides a method of treating a subject with acute lymphoblastic leukemia (ALL), by administering a drug combination comprising a CD19 antibody drug conjugate (CD19-ADC) and the chemotherapeutic drugs cyclophosphamide, vincristine, doxorubicin, and dexamethasone. The CD19-ADC is preferably SGN-CD19A, i.e., a humanized hBU12 antibody conjugated to a maleimidocaproyl monomethyl auristatin F (mcMMAF) molecule. In one embodiment, the subject has relapsed or refractory ALL. In a further embodiment, the CD19-ADC is administered at a dosage between 0.5 and 6.0 mg/kg.

In one aspect, the present disclosure provides a method of treating a subject with acute lymphoblastic leukemia (ALL), by administering a drug combination consisting essentially of a CD19 antibody drug conjugate (CD19-ADC) and the chemotherapeutic drugs cyclophosphamide, vincristine, doxorubicin, and dexamethasone. The CD19-ADC is preferably SGN-CD19A, i.e., a humanized hBU12 antibody conjugated to a maleimidocaproyl monomethyl auristatin F (mcMMAF) molecule. In one embodiment, the subject has relapsed or refractory ALL. In a further embodiment, the CD19-ADC is administered at a dosage between 0.5 and 6.0 mg/kg.

In one aspect, the present disclosure provides a method of treating a subject with acute lymphoblastic leukemia (ALL), by administering a drug combination consisting of a CD19 antibody drug conjugate (CD19-ADC) and the chemotherapeutic drugs cyclophosphamide, vincristine, doxorubicin, and dexamethasone. The CD19-ADC is preferably SGN-CD19A, i.e., a humanized hBU12 antibody conjugated to a maleimidocaproyl monomethyl auristatin F (mcMMAF) molecule. In one embodiment, the subject has relapsed or refractory ALL. In a further embodiment, the CD19-ADC is administered at a dosage between 0.5 and 6.0 mg/kg.

### DEFINITIONS

The term “CD19” refers to “cluster of differentiation protein 19”, a human protein that is expressed on human B cells. The amino acid sequence of human CD19 is known and is disclosed, e.g., at NCBI Reference Sequence: NP_001171569.1.

A “disorder”, as used herein, and the terms “CD19-associated disorder” and “CD19-associated disease” refer to any condition that would benefit from treatment with a CD19-antibody drug conjugate (CD19-ADC), such as SGN-CD19A, as described herein. This includes chronic and acute disorders or diseases including those pathological conditions that predispose the mammal to the disorder in question. Non-limiting examples or disorders to be treated herein include CD19 expressing cancers, including hematological malignancies, benign and malignant tumors, leukemias and lymphoid malignancies, as well as inflammatory, angiogenic and immunologic disorders. Specific examples of disorders are disclosed infra.

B cell malignancies, also referred to as B-cell lineage malignancies, are treatable by the methods of the present invention. The term B cell malignancies include any malignancy that is derived from a cell of the B cell lineage.

The terms “treatment” and “therapy”, and the like, as used herein, are meant to include therapeutic or suppressive measures for a disease or disorder leading to any clinically desirable or beneficial effect, including, but not limited to, alleviation or relief of one or more symptoms, regression, slowing or cessation of progression of the disease or disorder. For example, treatment can include a decrease or elimination of a clinical or diagnostic symptom of a CD19-expressing disorder after the onset of the clinical or diagnostic symptom by administration of an anti-CD19 antibody or other CD19 binding agent to a subject. Treatment can be evidenced as a decrease in the severity of a symptom, the number of symptoms, or frequency of relapse.

Except when noted, the terms “subject” or “patient” are used interchangeably and refer to mammals such as human patients and non-human primates, as well as experimental animals such as rabbits, dogs, cats, rats, mice, and other animals. Accordingly, the term “subject” or “patient” as used herein means any mammalian patient or subject to which the CD19 binding agents of the invention can be administered. In preferred embodiments, the terms subject or patient are used to refer to human patients. Subjects of the present invention include those that have been diagnosed with a CD19 expressing cancer, including, for example, B cell lymphoma or B cell leukemia, including, but not limited to, non-Hodgkin lymphoma, chronic lymphocytic leukemia, and acute lymphoblastic leukemia. In certain embodiments, the subject will have a refractory or relapsed CD19 expressing cancer

A subject with a refractory CD19 expressing cancer is a subject who does not respond to therapy, i.e., the subject continues to experience disease progression despite therapy.

A subject with a relapsed CD19 expressing cancer is a subject who has responded to the therapy at one point, but has had a recurrence or further progression of disease following the response.

The term “effective amount” refers to the amount of a CD19-ADC, e.g., SGN-CD19A, that is sufficient to inhibit the occurrence or ameliorate one or more clinical or diagnostic symptoms of a CD19-associated disorder in a subject. An effective amount of an agent is administered according to the methods described herein in an “effective regimen.” The term “effective regimen” refers to a combination of amount of the agent and dosage frequency adequate to maintain high CD19 occupancy, which may accomplish treatment or prevention of a CD19-associated disorder. In a preferred embodiment, an effective regimen maintains near complete, e.g., greater than 90%, CD19 occupancy on CD19-expressing cells during dosing intervals.

The term “pharmaceutically acceptable” as used herein refers to those compounds, materials, compositions, and/or dosage forms that are, within the scope of sound medical judgment, suitable for contact with the tissues of human beings and animals without excessive toxicity, irritation, allergic response, or other problems or complications commensurate with a reasonable benefit/risk ratio. The term “pharmaceutically compatible ingredient” refers to a pharmaceutically acceptable diluent, adjuvant, excipient, or vehicle with which a CD19-ADC, e.g., SGN-CD19A is administered.

The term “pharmaceutically compatible ingredient” refers to a pharmaceutically acceptable diluent, adjuvant, excipient, or vehicle with which a CD19-ADC, e.g., SGN-CD19A, is administered.

As used herein, the term “about” denotes an approximate range of plus or minus 10% from a specified value. For instance, the language “about 20%” encompasses a range of 18-22%. As used herein, about also includes the exact amount. Hence “about 20%” means “about 20%” and also “20%.

### BRIEF DESCRIPTION OF THE DRAWINGS

FIG. 1 provides the structure of SGN-CD19A.

FIGS. 2A-2D demonstrate the dose response of SGN-19A or CVAD in xenograft models established from ALLpatient samples. FIGS. 2A and 2B show the dose response curves of SGN-CD19A in xenografts from donor 06343 (FIG. 2A) and from donor 90811 (FIG. 2B). FIGS. 2C and 2D show the dose response curves of CVAD in xenografts from donor 06343 (FIG. 2C) and from donor 90811 (FIG. 2D).

FIGS. 3A-3D show the response of xenografts established from ALL patient samples to SGN-CD19A, CVAD or the combination. Two dose levels of CVAD were assessed alone and incombination with SGN-CD19A. FIGS. 3A and 3B show the response of donor 06343 to high dose (FIG. 3A) and low dose (FIG. 3B) CVAD. FIGS. 3C and 3D show the response of donor 90811 to high dose (FIG. 3C) and low dose (FIG. 3D) CVAD.

FIGS. 4A and 4B show the results of treatment of xenografts established using NALM6 (FIG. 4B) or Rs411 (FIG. 4B) cell lines using SGN-CD19A, CVAD, or the combination, or single components of CVAD, alone or in combination with SGN-CD19A. The median survival for each group is also summarized in Tables 1 and 2.

FIG. 5 shows disease burden in xenografts established from ALL patient donor 90811 after treatment with SGN-CD19A, CVAD, or the combination, or single components of CVAD, alone or in combination with SGN-CD19A.

FIG. 6 shows disease burden in xenografts established from ALL patient donor 06343 after treatment with SGN-CD19A, CVAD, or the combination, or single components of CVAD, alone or in combination with SGN-CD19A.

FIG. 7 demonstrates the effect of the combination of SGN-CD19A and vincristine on NALM6 cells grown in vitro.

FIG. 8 demonstrates the effect of the combination of SGN-CD19A and doxorubicin on NALM6 cells grown in vitro.

### DETAILED DESCRIPTION

The present invention provides, inter alia, methods for treating acute lymphoblastic leukemia (ALL), in particular CD19 positive ALL. The present inventors have discovered that combination therapy with two different classes of anticancer compounds, antibody-drug conjugate compounds and chemotherapeutic agents, can improve a therapeutic benefit for subjects suffering from ALL. In particular, the present inventors have found that combination therapy with vincristine and an anti-CD19 antibody conjugated to an auristatin compound provides synergistic therapeutic effects in the treatment of ALL. Similarly, the present inventors have found that combination therapy with doxorubicin and an anti-CD19 antibody conjugated to an auristatin compound provides synergistic therapeutic effects in the treatment of ALL. Before the advent of the present invention, it could not have been expected that a chemotherapeutic agent and an anti-CD30 antibody conjugated to an auristatin compound would have a synergistic effect in the treatment of ALL.

For clarity of disclosure, and not by way of limitation, the detailed description of the invention is divided into the subsections which follow.

### CD19-ADC

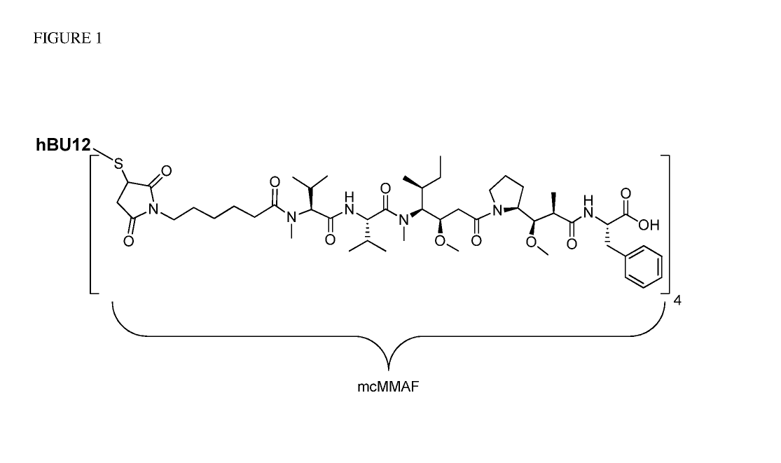

A CD19-antibody drug conjugate (CD19-ADC) includes an antibody specific for the human CD19 protein conjugated to a cytotoxic agent. SGN-CD19A is a CD19ADC produced by the conjugation of the drug-linker intermediate maleimidocaproyl monomethyl auristatin F (mcMMAF) to the humanized antibody hBU12 (FIG. 1). The points of attachment are cysteines produced by reduction of inter-chain disulfides. SGN-CD19A has an average of four drugs per antibody molecule.

Methods of making the hBU12 antibody are disclosed, e.g., at U.S. Pat. No. 7,968,687. The amino acid sequence of the light chain variable region of hBU12 is provided herein as SEQ ID NO:1. The amino acid sequence of the heavy chain variable region of hBU12 is provided herein as SEQ ID NO:2. hBU12 is an IgG1 antibody and the variable regions are joined to human heavy and light constant regions. U.S. Pat. No. 7,968,687 also provides methods for the synthesis of mcMMAF and its conjugation to hBU12.

SGN-CD19A, therefore, is an ADC that delivers mcMMAF to CD19-positive cells. mcMMAF is a tubulin-binding molecule. SGN-CD19A has a proposed multi-step mechanism of action initiated by binding to its target on the cell surface and subsequent internalization. After cell surface binding, internalization, and trafficking of SGN-CD19A through the endocytic pathway, proteolytic degradation of hBU12 in the lysosomes releases the cysteine adduct of the drug linker in the form of cys-mcMMAF, which then becomes available for tubulin binding. See, e.g., Doronina et al., Nat Biotechnol 21:778-84 (2003) and Doronina et al., Bioconjug Chem 17: 114-24 (2006). cys-mcMMAF and mcMMAF are used interchangeably herein. Binding of the released drug to tubulin disrupts the cellular microtubule network, leading to G2/M phase cell cycle arrest and subsequent onset of apoptosis in the targeted cell.

### Combination of Chemotherapy Agents and SGN-CD19A to Treat ALL

ALL can be treated using a combination of chemotherapeutic agents known as CVAD, i.e., a combination of Cyclophosphamide, Vincristine sulfate, doxorubicin hydrochloride (Adriamycin), and Dexamethasone. Cyclophosphamide is a synthetic alkylating agent chemically related to the nitrogen mustards. Vincristine is a natural alkaloid isolated from the plant Vinca rosea Linn with antimitotic and antineoplastic activities. Vincristine binds to microtubules and spindle proteins in S phase of the cell cycle and interferes with the formation of the mitotic spindle, thereby arresting tumor cells in metaphase. Doxorubicin is an anthracycline antibiotic with antineoplastic activity. Dexamethasone is a steroid and can be used as a direct chemotherapeutic agent in certain haematological malignancies, including ALL. Treatment of ALL using CVAD or hyper-CVAD is known to those of skill and is described at, e.g. Thomas et al., Blood 104:1624-1630 (2004).

This disclosure demonstrates that the combination of CVAD chemotherapy with SGN-CD19A can be given to subjects at levels that inhibit cancer cell growth, while at the same time are tolerated by the subject. Further, CVAD and SGN-CD19A can be effectively administered to achieve antitumor therapeutic effects as a combination at lower levels than either when administered alone. Thus, the combination of SGN-CD19 and CVAD is synergistic.

In combination with CVAD, SGN-CD19A is administered at a lower level than when used as a single agent. For example in combination with CVAD SGN-CD19A is administered at a dose between 0.1 and 6.0 mg/kg. Other appropriate dose ranges of SGN-CD19A in combination with CVAD are 0.1 to 4.0 mg/kg, 0.5 to 3.0 mg/kg, and 0.5 to 2.0 mg/kg. In combination with SGN-CD19A, CVAD can also be administered at levels that are less than typical, e.g., one half or one quarter, or one tenth of the usual dose.

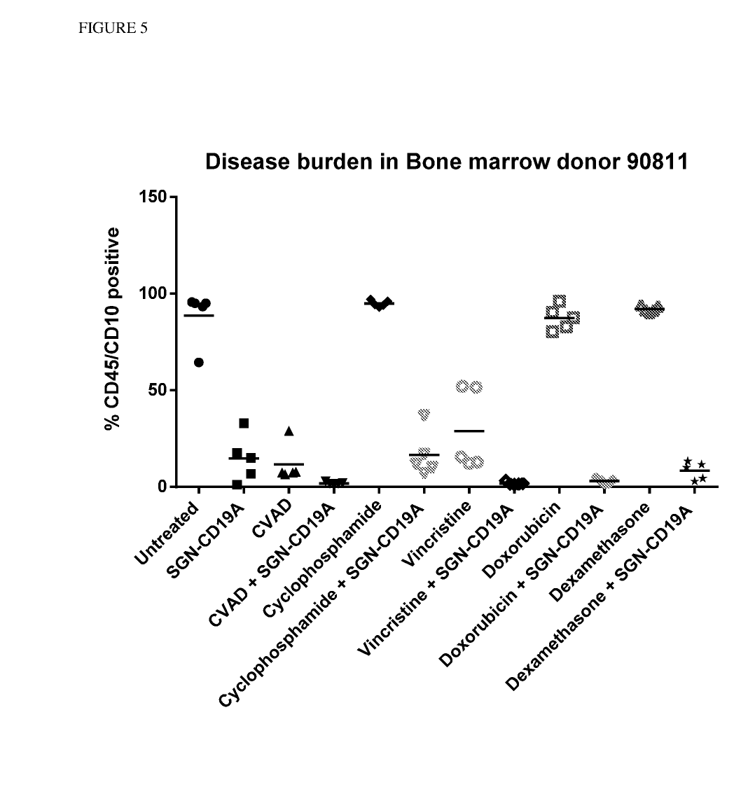

This disclosure also demonstrates that some of the components of CVAD, e.g., vincristine and doxorubicin, can be administered with SGN-CD19A and decreased tumor cell growth to levels similar to those of the SGN-CD19A plus CVAD combination. See, e.g., FIGS. 5-8. Thus, combinations of SGN-CD19A plus chemotherapeutic agents can be selected that use fewer agents and potentially, result in fewer side effects for patients.

In combination with vincristine, SGN-CD19A is administered at a lower level than when used as a single agent. Thus, the combination of SGN-CD19A and vincristine is synergistic. For example in combination with vincristine, SGN-CD19A is administered at a dose between 0.1 and 6.0 mg/kg. Other appropriate dose ranges of SGN-CD19A in combination with vincristine are 0.1 to 4.0 mg/kg, 0.5 to 3.0 mg/kg, and 0.5 to 2.0 mg/kg. In combination with SGN-CD19A, vincristine can also be administered at levels that are less than typical, e.g., one half or one quarter, or one tenth of the usual dose.

In combination with doxorubicin, SGN-CD19A is administered at a lower level than when used as a single agent. Thus, the combination of SGN-CD19A and doxorubicin is synergistic. For example in combination with doxorubicin, SGN-CD19A is administered at a dose between 0.1 and 6.0 mg/kg. Other appropriate dose ranges of SGN-CD19A in combination with doxorubicin are 0.1 to 4.0 mg/kg, 0.5 to 3.0 mg/kg, and 0.5 to 2.0 mg/kg. In combination with SGN-CD19A, doxorubicin can also be administered at levels that are less than typical, e.g., one half or one quarter, or one tenth of the usual dose.

Vincristine and SGN-CD19A can also be administered in combination with one or two additional components of CVAD. For example, SGN-CD19A can be administered in combination with vincristine and doxorubicin and cyclophosphamide or in combination with vincristine and cyclophosphamide and dexamethasone, or in combination with vincristine and doxorubicin and dexamethasone, or in combination with vincristine and doxorubicin, or in combination with vincristine and cyclophosphamide, or in combination with vincristine and dexamethasone. Other combination with SGN-CD19A and CVAD components include, e.g., SGN-CD19A combined with doxorubicin and cyclophosphamide or SGN-CD19A combined with doxorubicin and dexamethasone.

### Administration

SGN-CD19A and a CVAD regimen, or vincristine, or doxorubicin are administered in such a way that they provide a synergistic effect in the treatment of ALL in a patient. Administration can be by any suitable means provided that the administration provides the desired therapeutic effect. In preferred embodiments, SGN-CD19A and CVAD, or SGN-CD19A and vincristine, or SGN-CD19A and doxorubicin are administered during the same cycle of therapy, e.g., during one cycle of therapy, e.g., a three or four week time period, both SGN-CD19A and the specified chemotherapeutic drug(s) are administered to the subject.

The dosage of the antibody-drug conjugate compound administered to a patient with ALL will also depend on frequency of administration. The present invention contemplates antibody-drug conjugate compound delivery once during the treatment cycle or by a split delivery. CVAD is frequently administered in split doses, e.g., hyper CVAD and this administration can be used in the methods of the invention.

The present invention encompasses embodiments wherein SGN-CD19A will be administered in a dose range of 0.1 mg/kg to 2.7 mg/kg of the subject's body weight per dose, 0.2 mg/kg to 1.8 mg/kg of the subject's body weight per dose, 0.2 mg/kg to 1.2 mg/kg of the subject's body weight per dose, 0.4 mg/kg to 1 mg/kg of the subject's body weight per dose, 1.0 mg/kg to 1.5 mg/kg of the subject's body weight per dose, and 0.5 mg/kg to 1 mg/kg of the subject's body weight per dose. Other ranges are encompassed by the present invention as long as they produce the desired synergistic result.

The present invention encompasses treatment schedules wherein the total dosage of SGN-CD19A, administered to a patient with ALL will be, for example, 0.1 mg/kg to 6 mg/kg, 0.1 mg/kg to 4 mg/kg, 0.1 mg/kg to 3.2 mg/kg, or 0.1 mg/kg to 2.7 mg/kg of the subject's body weight over a treatment cycle, e.g., a 3 or 4 week time period. In some embodiments, the total dosage of the antibody-drug conjugate compound administered to a patient with ALL will be, for example about 0.6 mg/kg to about 6 mg/kg, about 0.6 mg/kg to about 4 mg/kg, about 0.6 mg/kg to about 3.2 mg/kg, about 0.6 mg/kg to about 2.7 mg/kg, or even about 1.5 mg/kg to about 3 mg/kg over a treatment cycle, e.g., a 3 or 4 week time period. In some embodiments, the dosage will be about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, or about 3.8 mg/kg of the subject's body weight over the treatment cycle, e.g., a 3 or 4 week time period. The present invention contemplates administration of the drug for one or more treatment cycles, for example, 1, 2, 3, 4, 5, 6, or more, treatment cycles. In some embodiments, there will be periods of rest between one or more of the treatment cycles. For example, in some embodiments, there will be a period of rest between the second and third treatment cycle but not the first and second treatment cycle. In another embodiment, there might be a period of rest between the first and second treatment cycle but not the second and third treatment cycle. Dosing schedules include, for example, administering SGN-CD19A once during a treatment schedule, e.g., on day 1 of a 21 day cycle, twice during a treatment cycle, e.g., on days 1 and 15 of a 28 day cycle, and three times during a treatment cycle, e.g., on days 1, 8 and 15 of a 28 day cycle. Other dosage schedules are encompassed by the present invention.

The present invention encompasses treatment schedules wherein SGN-CD19A is administered once during a treatment cycle, e.g., a 3 or 4 week time period. For example, in some embodiments, the antibody-drug conjugate will be administered on the third week of a 3 or 4 week treatment cycle, e.g., on day 21 of a three or four week cycle. In some embodiments, the SGN-CD19A will be administered on day 1 of a 3 or 4 week treatment cycle, or on any other day of a three or four week treatment cycle. In some such embodiments, the dosage of SGN-CD19A administered to a patient with ALL will typically be, for example, 0.1 mg/kg to 6 mg/kg of the subject's body weight over the treatment cycle, e.g., a 3 or 4 week time period. More typically, the dosage will be 0.1 mg/kg to 4 mg/kg, 0.1 mg/kg to 3.2 mg/kg, 0.1 mg/kg to 2.7 mg/kg, 1 mg/kg to 2.7 mg/kg, 1.5 mg/kg to 2.7 mg/kg, or 1.5 mg/kg to 2 mg/kg of the subject's body weight over the treatment cycle, e.g., a 3 or 4 week time period. In some embodiments, the total dosage of SGN-CD19A administered to a patient with ALL will be, for example about 0.6 mg/kg to about 6 mg/kg, about 0.6 mg/kg to about 4 mg/kg, about 0.6 mg/kg to about 3.2 mg/kg, about 0.6 mg/kg to about 2.7 mg/kg, or even about 1.5 mg/kg to about 3 mg/kg over a treatment cycle, e.g., a 3 or 4 week time period. In some embodiments, the dosage will be about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, or about 3.8 mg/kg of the subject's body weight over the treatment cycle.

In other embodiments SGN-CD19A will be administered more than once during a treatment cycle. For example, in some embodiments, SGN-CD19A will be administered weekly for three consecutive weeks in a three or four week treatment cycle. For example, in some embodiments, SGN-CD19A will be administered on days 1, 8, and 15 of each 28 day treatment cycle. In some such embodiments, the dosage SGN-CD19A administered to a patient with ALL can be, for example, 0.1 mg/kg to 6 mg/kg, 0.1 mg/kg to 4 mg/kg, 0.1 mg/kg to 3.2 mg/kg, or 0.1 mg/kg to 2.7 mg/kg of the subject's body weight over the treatment cycle. In some embodiments, the total dosage of SGN-CD19A administered to a patient with ALL will be, for example about 0.6 mg/kg to about 6 mg/kg, about 0.6 mg/kg to about 4 mg/kg, about 0.6 mg/kg to about 3.2 mg/kg, about 0.6 mg/kg to about 2.7 mg/kg, or even about 1.5 mg/kg to about about 3 mg/kg over the treatment cycle. In some embodiments, the dosage will be about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg , about 3.9 mg/kg or about 4.0 mg/kg of the subject's body weight over the treatment cycle. In some embodiments, the dosage will generally be 0.1 to 5 mg/kg of the subject's body weight, 0.1 mg/kg to 3.2 mg/kg of the subject's body weight, even more typically, 0.1 mg/kg to 2.7 mg/kg, 0.2 mg/kg to 1.8 mg/kg, 0.2 mg/kg to 1.2 mg/kg, 0.2 mg/kg to 1 mg/kg, 0.4 mg/kg to 1 mg/kg, or 0.4 mg/kg to 0.8 mg/kg of the subject's body weight on days 1, 8, and 15 of each 28 day cycle. In some embodiments, the dosage will be about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg/about 1.4 mg/kg, or about 1.5 mg/kg of the subject's body weight on days 1, 8, and 15 of each 28 day cycle.

In even other embodiments SGN-CD19A will be administered every two weeks in a four week treatment cycle. For example, in some embodiments, SGN-CD19A will be administered on days 1 and 15 of each 28 day treatment cycle. In some such embodiments, the dosage of SGN-CD19A administered to a patient with ALL can be, for example, 0.1 mg/kg to 6 mg/kg, 0.1 mg/kg to 4 mg/kg, 0.1 mg/kg to 3.2 mg/kg, or 0.1 mg/kg to 2.7 mg/kg of the subject's body weight over the treatment cycle. In some embodiments, the total dosage of SGN-CD19A administered to a patient with ALL will be, for example about 0.6 mg/kg to about 6 mg/kg, about 0.6 mg/kg to about 4 mg/kg, about 0.6 mg/kg to about 3.2 mg/kg, about 0.6 mg/kg to about 2.7 mg/kg, or even about 1.5 mg/kg to about about 3 mg/kg over the treatment cycle. In some embodiments, the dosage will be about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, or about 3.8 mg/kg of the subject's body weight over the treatment cycle. In some embodiments, the dosage of the antibody-drug conjugate compound will generally be 0.1 mg/kg to 5 mg/kg of the subject's body weight, 0.1 mg/kg to 3.2 mg/kg of the subject's body weight, more typically 0.1 mg/kg to 2.7 mg/kg, even more typically 0.2 mg/kg to 1.8 mg/kg, 0.2 mg/kg to 1.2 mg/kg, 0.2 mg/kg to 1.5 mg/kg, 1 mg/kg to 1.5 mg/kg, or 0.5 to 1.2 mg/kg, of the subject's body weight on days 1 and 15 of each 28 day cycle. In some embodiments, the dosage will be about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, or about 1.8 mg/kg of the subject's body weight on days 1 and 15 of each 28 day cycle.

It will be readily apparent to those skilled in the art that other SGN-CD19A doses or frequencies of administration that provide the desired synergistic effect in combination with CVAD, vincristine, or doxorubicin are suitable for use in the present invention.

Administration of SGN-CD19A and CVAD or a component of CVAD, e.g., vincristine or doxorubicin, can be on the same or different days provided that administration provides the desired therapeutic effect. In some embodiments of the present invention, administration of SGN-CD19A and CVAD or a CVAD component will be on the same and/or different days, e.g, the SGN-CD19A will be administered on day 1 of a 21 day cycle and CVAD or a CVAD component will be administered on day 1 and 8 or day 1 and 15 of the 21 day cycle. Alternative treatment schedules are encompassed by the present invention as long as they produce the desired result.

In some embodiments, CVAD or a component of CVAD, e.g., vincristine or doxorubicin, will be administered at levels currently indicated in the art for the treatment of ALL or at lower or higher levels than those currently indicated in the art for the treatment of ALL provided that such dosage provides the desired therapeutic effect. Embodiments of the present invention include, for example, those wherein the CVAD or a component of CVAD, e.g., vincristine or doxorubicin, is administered at about the MTD, maximum tolerated dose. The present invention contemplates administration of CVAD or a component of CVAD, e.g., vincristine or doxorubicin, for one or more treatment cycles, for example, 1, 2, 3, 4, 5, 6, or more treatment cycles. It will be understood that any of the dose ranges indicated herein for treatment with CVAD or a component of CVAD, e.g., vincristine or doxorubicin, can be combined with any of the dose ranges indicated herein for treatment SGN-CD19A provided that administration provides the desired therapeutic effect.

In some particularly preferred examples of the present invention, administration of a synergistic amount of the therapeutic agents encompasses SGN-CD19A once during the treatment cycle (e.g., a 21 or 28 day treatment cycle) in a range of about 0.5 to about 6.0 mg/kg, about 0.6 mg/kg to about 4.0 mg/kg, about 0.6 mg/kg to about 2 mg/kg, about 0.6 mg/kg to about 1 mg/kg, about 0.8 mg/kg to about 4.0 mg/kg, about 0.8 mg/kg to about 2.0 mg/kg, about 1 mg/kg to about 2.7 mg/kg, about 1.5 mg/kg to about 2.7 mg/kg, or even more preferably about 1.0 mg/kg to about 2 mg/kg or about 1.5 mg/kg to about 2 mg/kg of the subject's body weight in combination with administering CVAD or a component of CVAD, e.g., vincristine or doxorubicin, at standard dosing schedules known in the art.

In embodiments of the present invention wherein treatment comprises administration of SGN-CD19A and CVAD or a component of CVAD, e.g., vincristine or doxorubicin, administration of SGN-CD19A can be on the same or different days as administration of the chemotherapeutic regimen provided that administration provides the desired therapeutic effect. Methods of administering CVAD or a component of CVAD, e.g., vincristine or doxorubicin, in a chemotherapeutic regimen for the treatment of ALL are known. Embodiments of the present invention include those wherein the drugs are administered at the levels currently indicated in the art for the treatment of ALL. Embodiments of the present invention include those wherein the drugs are administered at lower or higher levels than currently indicated in the art for the treatment of ALL provided that administration provides the desired synergistic effect. In certain instances, dosage levels can be reduced when SGN-CD19A is combined with CVAD or a component of CVAD, e.g., vincristine or doxorubicin.

In some particularly preferred examples of the present invention, administration of a synergistic amount of the therapeutic agents encompasses administering SGN-CD19A in a total range of about 0.5 mg/kg to about 6 mg/kg, about 0.6 mg/kg to about 5 mg/kg, about 0.6 mg/kg to about 2.7 mg/kg, about 0.8 mg/kg to about 2.7 mg/kg, about 1 mg/kg to about 5 mg/kg, about 1 mg/kg to about 4 mg/kg, about 1 mg/kg to about 3.5 mg/kg, about 1.5 mg/kg to about 3. 5 mg/kg or even about 1.8 mg/kg to about 2.5 mg/kg over a 21 or 28 day treatment cycle, irrespective of the dosing schedule, in combination with administering CVAD or a component of CVAD, e.g., vincristine or doxorubicin, at standard dosing schedules known in the art.

### Subjects

The methods of the present invention encompass administering combination therapy to a subject for the treatment of CD19 positive acute lymphocytic leukemia (ALL).

The subjects to be treated with the methods of the present invention are those that have been diagnosed with ALL or are suspected of having ALL. Diagnosis can be by methods known in the art, including, identification of immature white blood cells (lymphoblasts) in peripheral blood or bone marrow.

The methods of the present invention encompass treating a subject who is newly diagnosed and has not previously been treated for ALL.

The methods of the present invention also can be used to treat subjects with refractory and/or relapsed ALL. A subject with refractory ALL is a subject who does not respond to therapy for ALL, i.e., the subject continues to experience disease progresssion despite therapy. A subject with relapsed ALL is a subject who has responded to therapy for ALL at one point, but has had a reoccurrence or further progression of disease following the response.

The methods of the present invention also encompass treating a subject who has previously undergone a stem cell transplant.

### EXAMPLES

The following examples are offered to illustrate, but not to limit the claimed invention.

### Materials and Methods

Establishment of cell line xenografts: NALM-6 (DSMZ, Braunschweig, Germany) and RS4; 11 (ATCC, Manassas, Va.) cell lines were cultured in RPMI 1640 media, supplemented with Heat Inactivated Fetal Bovine serum (10%) and Penicillin/Streptomycin(1%) (Gibco, Grand Island, N.Y.). Cells were maintained in a humidified atmosphere at 37° C. and 5% CO₂. For NALM-6 cell implantation, 1.0×10⁵ cells in 200 ul PBS were injected into the tail vein of female C.B-17 SCID (Harlan Laboratories, Livermore, Calif.) mice. Mice were randomly assigned to treatment groups and treatment was given seven days post cell implant. For RS4; 11 cell implantation, 1.8×10⁶ cells in 200 ul PBS were implanted into the tail vein of female C.B-17 SCID mice. Mice were randomly assigned to treatment groups. Treatment was given one day post cell implant. Treatment in the NALM-6 consisted of a single dose of SGN-CD19A at 1.0 mg/kg; in RS4; 11, SGN-CD19A was given once every four days for four doses at 0.3 mg/kg. Cyclophosphamide, 30 mg/kg (Baxter, Deefield, Ill.), Vincristine, 0.375 mg/kg (Hospira, Lake Forest, Ill.), and Doxorubicin, 2.475 mg/kg (Pfizer, NY, NY) were combined and administered as a single dose via intravenous injection into the lateral tail vein. Dexamethasone, 15 mg/kg (APP Pharmaceuticals, Schaumberg, Ill.), was given daily for five days via intraperitoneal injection. For each xenograft model, mice were monitored daily and body weights were collected at least once per week. Mice were removed from experiment at the onset of clinical disease signs (hind limb weakness, piloerection, hunched posture) or weight loss in excess of 20% of initial body weight.

Establishment of patient-derived xenografts: NOD-scid IL2Rγ null mice were used as hosts for patient-derived xenografts. Twenty-four hours prior to cell implant, mice received 1.0 Gy irradiation using a Radsource 2000 irradiator (Radsource technologies, Suwanee, Ga.). Frozen bone marrow isolates of mononuclear cells from patients with acute lymphoblastic leukemia were obtained from ALLCELLS (Alameda, Calif.). Cells were thawed in a warm water bath. After thaw, RPMI-1640 media was slowly added to the bone marrow isolate suspension. The cells were washed with PBS and centrifuged to remove DMSO in freezing media. After centrifugation, the cells were suspended in PBS. For, implantation, 3.6×10⁶ cells were injected into the tail vein of female NOD-scid IL2Rγ null mice designated as donor, or passage 0 mice. Approximately 7 weeks after implant, mice were euthanized. Bone marrow and spleen were collected using sterile technique. Spleens were mechanically disrupted using a sterile syringe end and mesh filter. Connective tissues from spleens were filtered out using 40um filters. Splenocytes and bone marrow cells were treated with red blood cell buffer lysis (BD lysis buffer, BD biosciences, San Jose, Calif.) for 15 minutes at room temperature. A sample of the cells was utilized for confirmation of engraftment. The percentage of blast cells was determined by the number of CD45 FITC, CD19 PE-Cy.5 (BD Biosciences, San Jose, Calif.) CD10 APC (Biolegend, San Diego, Calif.) triple positive cells via flow cytometry (FACScalibur, BD Biosciences, San Jose, Calif.). The majority of the cells (passage 1) were placed into freezing media (90% Heat inactivated fetal bovine serum and 10% DMSO). Subsequent xenograft experiments utilized passage 1 cells. Cells were implanted in the same manner done for donor mice (passage 0 mice). Starting 14-21 days post implant, 2-3 sentinel mice were euthanized to determine the percentage blast cells in the bone marrow. Once the percentage of blast cells was at 25-50% of the bone marrow mononuclear cells mice were randomly placed into treatment groups. SGN-CD19A and non-specific ADC (also called h00-1269) were administered as a single intraperitoneal injection at 1.0, 3.0, or 10.0 mg/kg dose. Cyclophosphamide at 15 or 30 mg/kg (Baxter, Deefield, Ill.), Vincristine at 0.188 or 0.375 mg/kg (Hospira, Lake Forest, Ill.), and Doxorubicin at 1.24 or 2.475 mg/kg (Pfizer, NY, NY) were combined and administered as a single dose via intravenous injection into the lateral tail vein. Dexamethasone at 7.5 or 15 mg/kg (APP Pharmaceuticals, Schaumberg, Ill.) was given daily via intraperitoneal injection for five days. At pre-determined times post treatment, mice were euthanized for collection of bone marrow and determination of blast cell percentage via flow cytometry.

Isobolograms: Cells were plated at 5000 per well in 384 well plates using Fluid-X liquid handler (Fluid-X, Boston, Mass.). SGN-CD19A and Vincristine were plated separately and serial diluted in 2×96 well plates per drug. SGN-CD19A and Vincristine were added to 384 well plates, either alone or in combination at range above and below the IC₅₀ for each single agent using the Hamilton STAR automation robotics (Hamilton, Reno, Nev.). Cells were kept at 37° C. for 96 hours. Cell viability readout was performed using the CellTiter-Glo assay (Promega, Madison, Wis.). Luminescence was measured using the Envision plate reader (Perkin Elmer, Waltham, Mass.). Activity was determined by the luminescence of treated cells compared to that of untreated cells.

### Results

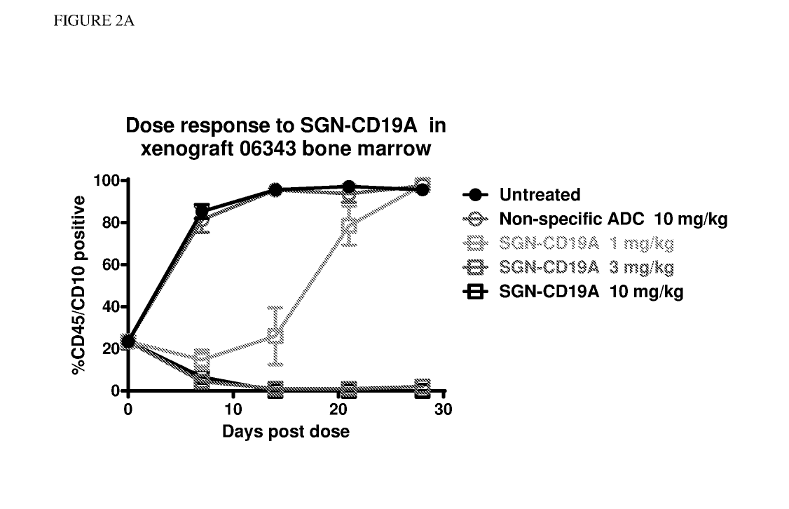

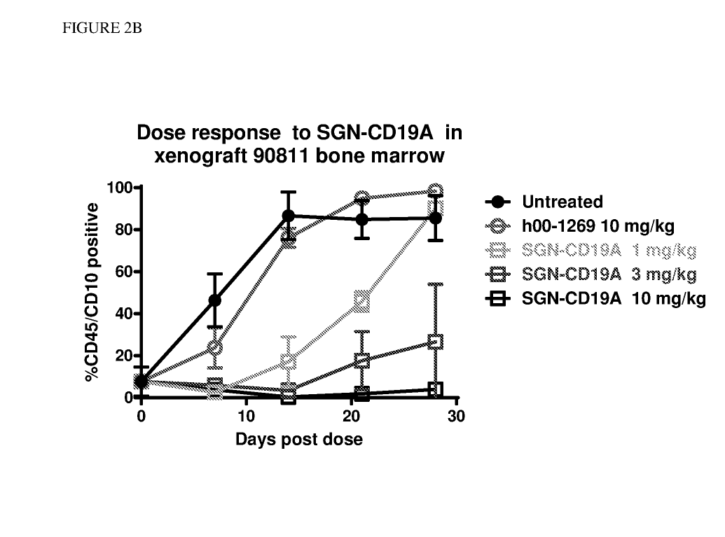

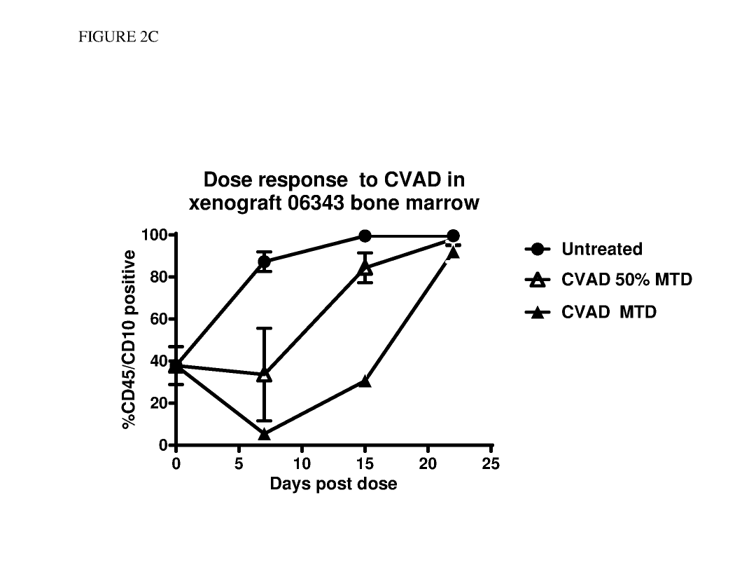

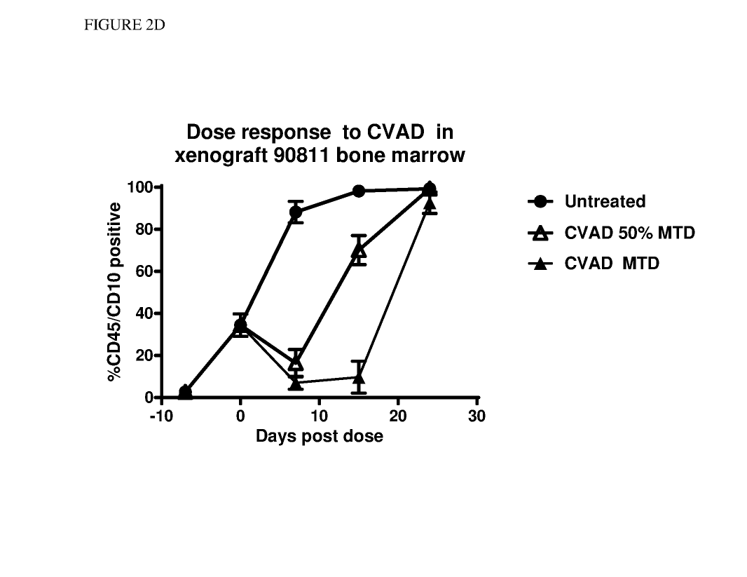

The response of human patient-derived ALL cells to single agent SGN-CD19A or CVAD was determined in mouse xenograft models. Bone marrow isolates from patients with ALL (donor 06343 and donor 90811) were used to establish xenografts in NOD-scid IL2Rgamma^{null} mice. After engraftment, patient-derived bone marrow cells were frozen and are referred to as passage 1 cells. Passage 1 cells were implanted into mice and assessed for presence of blast cells (CD45/CD10/CD19 positive cells). After blast cells were between 25-50% of the bone marrow, mice were placed in treatment groups (n=3 mice per time point). Mice were given either 1, 3, or 10 mg/kg of SGN-CD19A or 10 mg/kg of a non-specific ADC conjugated to MMAF (also called h00-1269). A maximum tolerated dose (MTD) of CVAD had previously been determined in the same mouse strain. Mice were given either CVAD at the MTD or at 50% of the MTD. Results are shown in FIGS. 2A-2D. For both patient samples, increasing amounts of SGN-CD19A led to decreasing amounts of blast cells in the bone marrow. At 50% of the MTD for CVAD, the response to drug was less than that of the MTD for both patient samples.

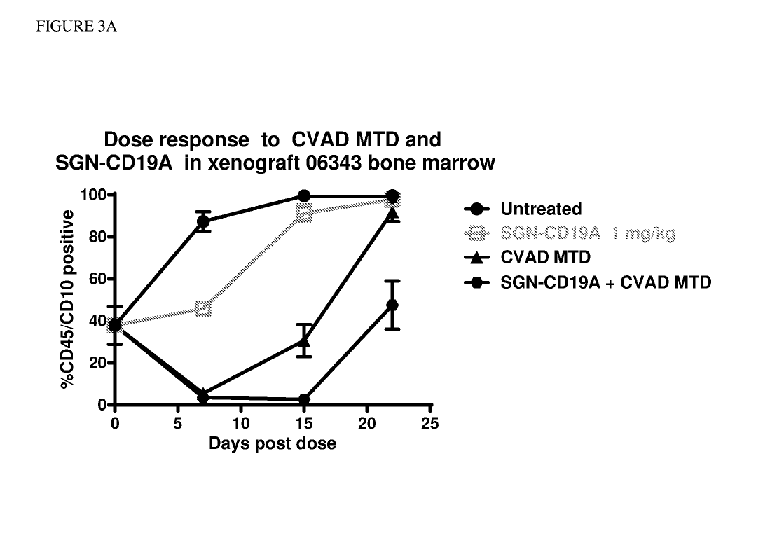

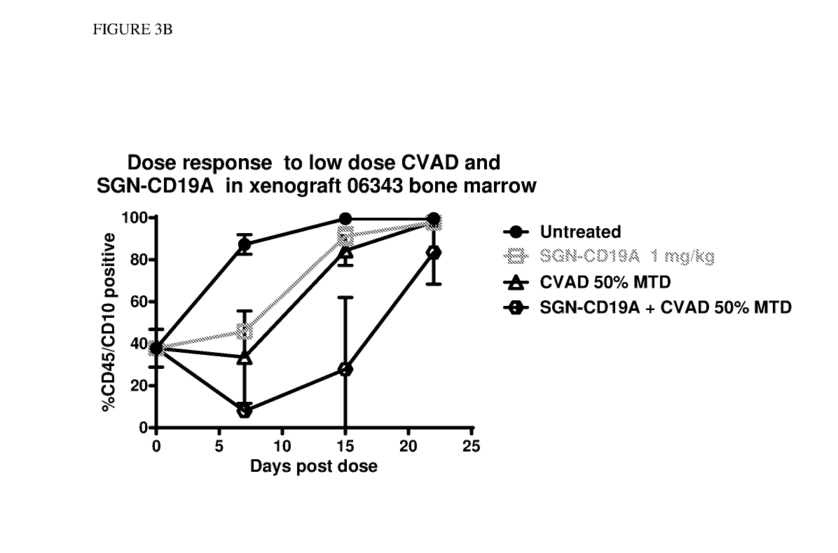

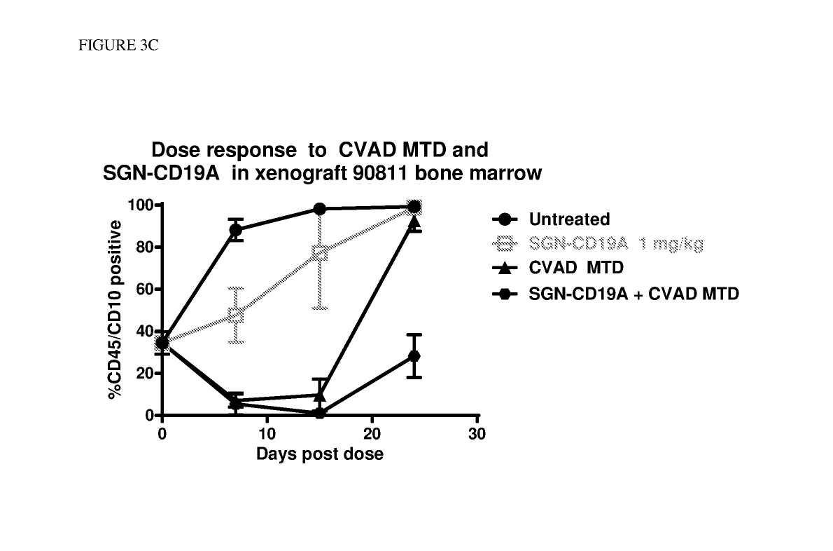

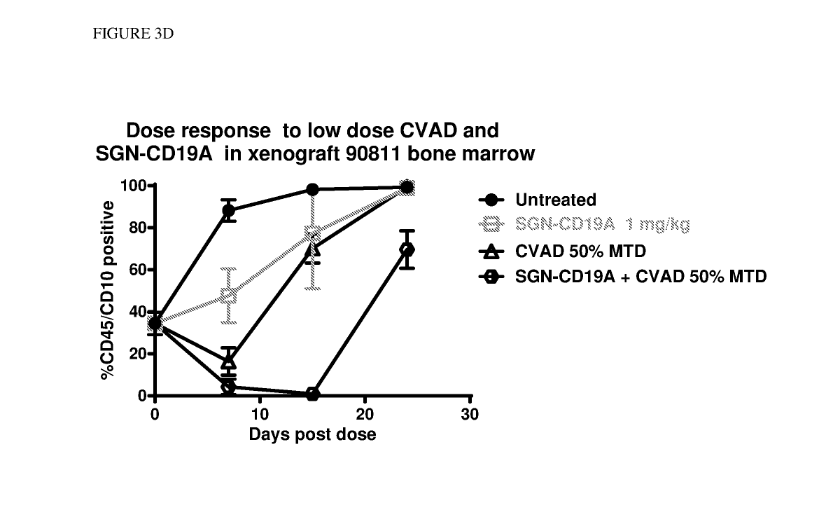

The response of human patient-derived ALL cells to the combination of SGN-CD19A and CVAD was also determined in the mouse xenograft model. Patient samples were engrafted as described above. Mice were not treated or were administered SGN-CD19A alone (1 mg/kg), CVAD at the MTD, or CVAD at 50% MTD, or a combination of SGN-19A (1 mg//kg) with CVAD at the MTD or a combination of SGNCD19A with CVAD at 50% MTD. Results are shown in FIGS. 3A-3D and each time point represents a group of three mice. In both patient samples, the combination of CVAD plus SGN-CD19A was significantly better than either SGN-CD19A or CVAD given alone.

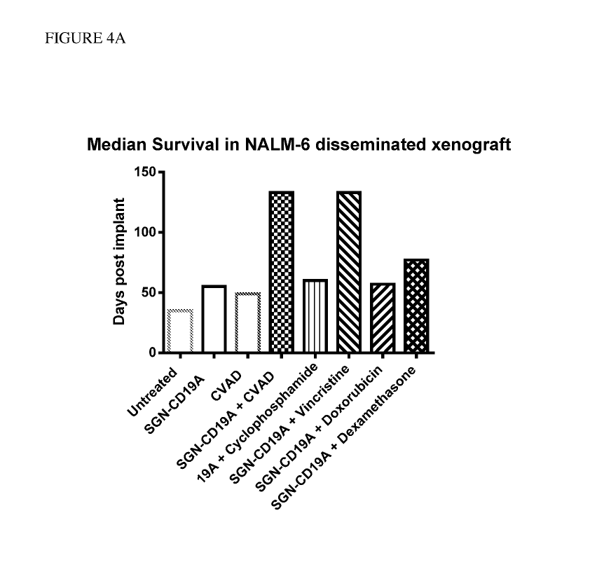

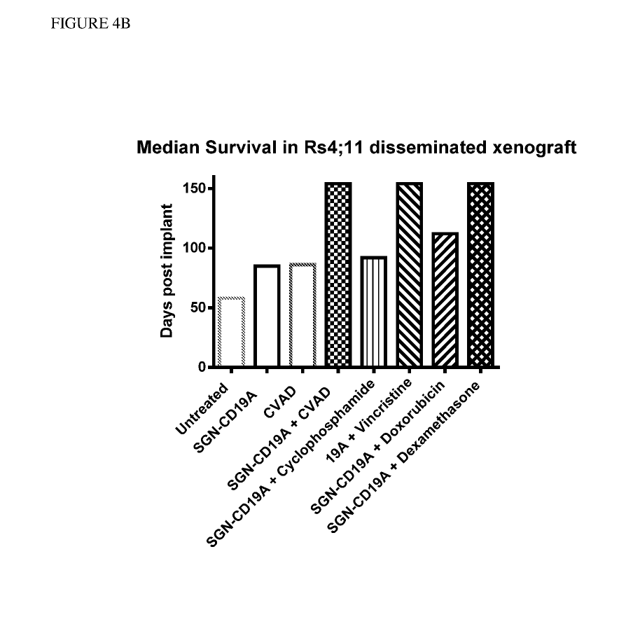

Cell line-derived xenografts were used to investigate the median survival time for the combination of SGN-CD19A and CVAD, as well as SGN-CD19A and each of the components of CVAD. Either NALM-6 or Rs4; 11 cells were implanted into CB-17 SCID mice. Ten mice were assigned to each treatment group. After signs of clinical disease developed (RS;411 cells-1 day post implant, NALM6 cells-7 days post-implant), mice were administered one of the following: no treatment, SGN-CD19A, CVAD, SGN-CD19A+CVAD, SGN-CD19A+cyclophosphamide, SGN-CD-19A+vincristine, SGN-CD19A+doxorubicin, or SGN-CD19A+dexamethasone. SGN-CD19A and CVAD were administered at sub-optimal doses. Results are shown in FIGS. 4A and 4B and Tables 1 and 2. For the NALM-6 experiment, surviving mice were sacrificed at 133 days. As a result, median survival time for the groups treated with SGN-CD19A+CVAD and SGN-CD19A+vincristine was not determined but was longer than 133 days (FIG. 4A and Table 1). For the Rs4; 11 experiment, the experiment was terminated on day 154. As a result, median survival time for the groups treated with SGN-CD19A+CVAD, SGN-CD19A+vincristine, and SGN-CD19A+dexamethasone was not determined but was longer than 154 days (FIG. 4B and Table 2). For both cell lines, the combination of SGN-CD19A and CVAD resulted in longer survival, than for either one alone. Surprisingly, the combination of SGN-CD19A, which releases the microtubule-disrupting agent cys-mcMMAF, with vincristine, which is also a microtubule-disrupting agent, resulted in a survival benefit similar to that seen for the SGN-CD19A plus CVAD combination. The combination of SGN-CD19A plus dexamethasone resulted in better survival benefits compared to SGN-CD19 or dexamethasone alone in both the NALM-6 and RS4; 11 models. In addition, the combination of SGN-CD19A plus doxorubicin also resulted in a survival benefit compared to SGN-CD19A or doxorubicin alone in the NALM-6 model.

TABLE 1
Median Survival in NALM-6 Disseminated Xenograft

| Treatment | Median Survival (days) |
|---|---|
| Untreated | 35 |
| SGN-CD19A | 55 |
| CVAD | 49 |
| SGN-CD19A + CVAD | Undefined (at least 133) |
| Cyclophosphamide | 38 |
| SGN-CD19A + Cyclophosphamide | 57 |
| Vincristine | 47 |
| SGN-CD19A + Vincristine | Undefined (at least 133) |
| Doxorubicin | 44 |
| SGN-CD19A + Doxorubicin | 57 |
| Dexamethasone | 39 |
| SGNCD19A + Dexamethasone | 77 |

TABLE 2
Median Survival in Rs411 Disseminated Xenograft

| Treatment | Median Survival (days) |
|---|---|
| Untreated | 58 |
| SGN-CD19A | 85 |
| CVAD | 86 |
| SGN-CD19A + CVAD | Undefined (at least 154) |
| Cyclophosphamide | 58 |
| SGN-CD19A + Cyclophosphamide | 92 |
| Vincristine | 79 |
| SGN-CD19A + Vincristine | Undefined (at least 154) |
| Doxorubicin | 62 |
| SGN-CD19A + Doxorubicin | 112 |
| Dexamethasone | 80 |
| SGNCD19A + Dexamethasone | Undefined (at least 154) |

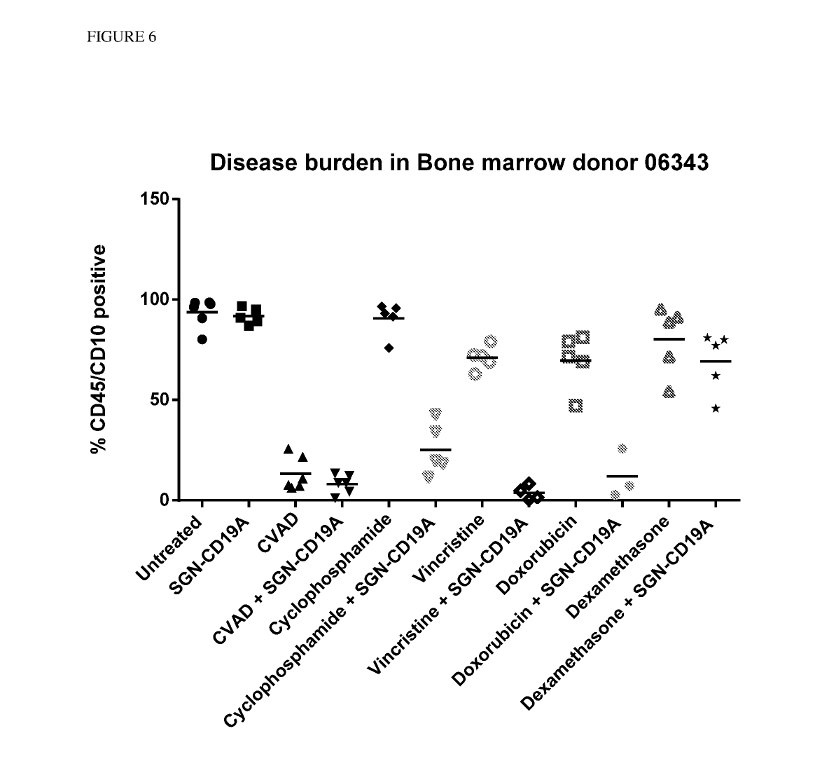

Xenografts from patient samples were also used to assess combination of SGN-CD19A and individual components of CVAD. Xenografts were established as described above. Tested agents were administered when the percentage of blast cells in sentinel animals was either 18% (Donor 90811) or 38% (Donor 06343). For donor 90811, bone marrow counts were performed on day 10 post-dose. For donor 06343, bone marrow counts were performed on day 14 post-dose. Results are show in FIGS. 5 and 6 and Tables 3 and 4. For both donors, the combination of SGN-CD19A and CVAD resulted in a lower percentage of blast cells in the bone marrow, than for either one alone. Vincristine+SGN-CD19A resulted in lower blast cell percentages in the bone marrow similar to SGN-CD19A+CVAD in both donors. Similarly, the doxorubicin+SGN-CD19A combination also decreased blast cell percentages in the bone marrow for both patient samples.

TABLE 3
Disease Burden in Bone Marrow Donor 90811

| | CD45/10% | |
|---|---|---|
| Treatment | Mean (STDEV) | |
| Untreated | 87.5 | (15.4) |
| SGN-CD19A | 18.2 | (11) |
| CVAD | 12.9 | (10.7) |
| SGN-CD19A + CVAD | 1.9 | (0.7) |
| Cyclophosphamide | 94.4 | (1.2) |
| SGN-CD19A + Cyclophosphamide | 17.7 | (13.5) |
| Vincristine | 32.2 | (22.7) |
| SGN-CD19A + Vincristine | 1.8 | (0.9) |
| Doxorubicin | 86.7 | (6.9) |
| SGN-CD19A + Doxorubicin | 2.6 | (1.1) |
| Dexamethasone | 91.8 | (1.6) |
| SGNCD19A + Dexamethasone | 9.5 | (4.6) |

TABLE 4
Disease Burden in Bone Marrow Donor 06343

| | CD45/10% | |
|---|---|---|
| Treatment | Mean (STDEV) | |
| Untreated | 92.7 | (7.6) |
| SGN-CD19A | 92.5 | (4.9) |
| CVAD | 13.7 | (9.2) |
| SGN-CD19A + CVAD | 8 | (5.2) |
| Cyclophosphamide | 90 | (9.6) |
| SGN-CD19A + Cyclophosphamide | 28.6 | (11.7) |
| Vincristine | 69.1 | (4.4) |
| SGN-CD19A + Vincristine | 3.3 | (4.3) |
| Doxorubicin | 69.2 | (15.5) |
| SGN-CD19A + Doxorubicin | 5 | (3.1) |
| Dexamethasone | 82.5 | (19) |
| SGNCD19A + Dexamethasone | 66.3 | (15.7) |

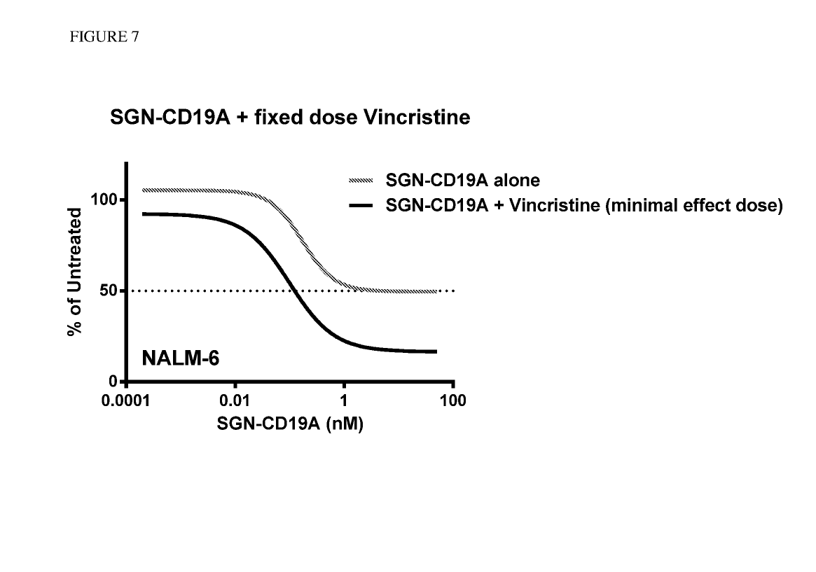

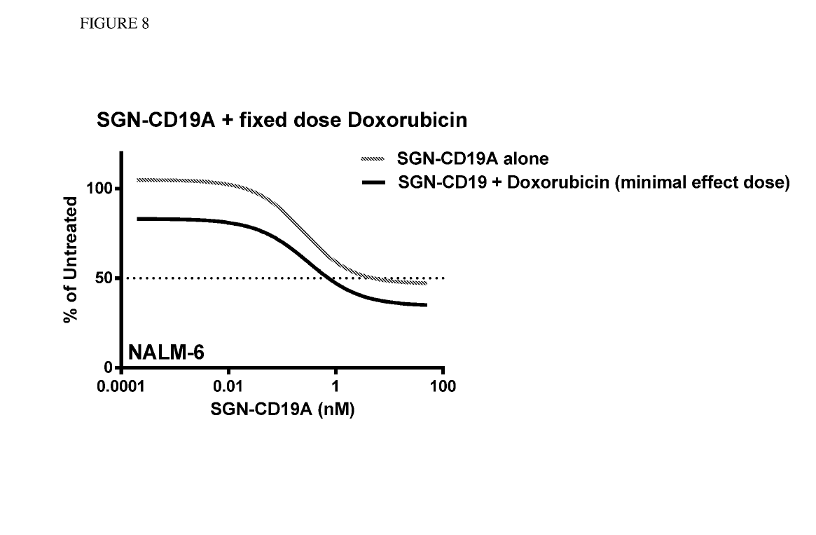

To verify the results of the in vivo SGN-CD19A plus vincristine or SGN-CD19A plus doxorubicin combinations, in vitro cytotoxicity experiments were performed. NALM6 cells were incubated with SGN-CD19A, vincristine only or a combination of SGN-CD19A plus vincristine. The concentration of each agent was serially diluted to include doses that would have no cytotoxicity to doses that would kill all cells. Results are shown in FIG. 7. Addition of a minimally effective dose of vincristine (approximately 5% cell kill as a single agent) to SGN-CD19A enhanced the cytotoxicity at all SGN-CD19A levels tested. NALM-6 cells were also incubated with a combination of SGN-CD19A and doxorubicin, in a similar manner. Results are shown in FIG. 8. Addition of doxorubicin to SGN-CD19A at a low effective dose (approximately 20% cell kill as a single agent) enhanced the cytotoxicity at all SGN-CD19A levels tested.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

INFORMAL SEQUENCE LISTING

hBU12 light chain variable region
SEQ ID NO: 1
eivltqspatlslspgeratlscsasssvsymhwyqqkpgqaprlliydt
sklasgiparfsgsgsgtdftltisslepedvavyycfqgsvypftfgqg
tkleikr
hBU12 heavy chain variable region
SEQ ID NO: 2
qvqlqesgpglvkpsqtlsltctvsggsistsgmgvgwirqhpgkglewi
ghiwwdddkrynpalksrvtisvdtsknqfslklssvtaadtavyycarm
elwsyyfdywgqgtlvtvss

What is claimed is:

1. A method of treating a subject with acute lymphoblastic leukemia (ALL), the method comprising administering to the subject a drug combination consisting essentially of a CD19 antibody drug conjugate (CD19-ADC) and vincristine, wherein the CD19-ADC comprises a humanized hBU12 antibody conjugated to a maleimidocaproyl monomethyl auristatin F (mcMMAF) molecule.

2. The method of claim 1, wherein the subject has relapsed or refractory ALL.

3. The method of claim 1, further comprising administration of cyclophosphamide, doxorubicin, or dexamethasone.

4. The method of claim 1, further comprising administration of cyclophosphamide, doxorubicin, and dexamethasone.

5. The method of claim 1, wherein the CD19-ADC is administered at a dosage between 0.5 and 6.0 mg/kg.

6. A method of treating a subject with acute lymphoblastic leukemia (ALL), the method comprising administering to the subject a drug combination consisting essentially of a CD19 antibody drug conjugate (CD19-ADC) and doxorubicin, wherein the CD19-ADC comprises a humanized hBU12 antibody conjugated to a maleimidocaproyl monomethyl auristatin F (mcMMAF) molecule.

7. The method of claim 6, wherein the subject has relapsed or refractory ALL.

8. The method of claim 6, further comprising administration of cyclophosphamide, vincristine, or dexamethasone.

9. The method of claim 6, further comprising administration of cyclophosphamide, vincristine and dexamethasone.

10. The method of claim 6, wherein the CD19-ADC is administered at a dosage between 0.5 and 6.0 mg/kg.

11. A method of treating a subject with acute lymphoblastic leukemia (ALL), the method comprising administering to the subject a drug combination consisting essentially of a CD19 antibody drug conjugate (CD19-ADC) cyclophosphamide, doxorubicin, dexamethasone and vincristine, wherein the CD19-ADC comprises a humanized hBU12 antibody conjugated to a maleimidocaproyl monomethyl auristatin F (mcMMAF) molecule.

12. The method of claim 11, wherein the subject has relapsed or refractory ALL.

13. The method of claim 11, wherein the CD19-ADC is administered at a dosage between 0.5 and 6.0 mg/kg.
